(19) United States
(12) Patent Application Publication
(10) Pub. No.: US 2004/0199148 A1
(43) Pub. Date: Oct. 7, 2004

(54) Method of ablating biological material with electromagnetic radiation delivered by an optical fiber

(76) Inventors: David Jon Haan, Sunnyvale, CA (US); Ronii Chris Mehl, Foresthill, CA (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/829,204

(22) Filed: Apr. 22, 2004

Related U.S. Application Data
(63) Continuation of application No. 10/124,711, filed on Apr. 18, 2002.
(60) Provisional application No. 60/285,013, filed on Apr. 19, 2001.

(51) Int. Cl. A61B 18/20

(52) U.S. Cl. 606/2; 606/3

(57) ABSTRACT

A device and a method for treating the human body is provided. The method includes generating electromagnetic radiation for example laser radiation, passing the electromagnetic radiation through a graded index optical fiber, the graded index optical fiber outputting the modified electromagnetic radiation and applying the modified electromagnetic radiation to the human body.

### PRIOR APPLICATIONS

This application is a continuation of U.S. application Ser. No. 10/124,711, entitled “METHOD OF ABLATING BIOLOGICAL MATERIAL WITH ELECTROMAGNETIC RADIATION DELIVERED BY AN OPTICAL FIBER”, filed Apr. 18, 2002 and incorporated herein by reference, which in turn claims benefit from prior U.S. provisional patent application serial No. 60/285,013 entitled “METHOD OF ABLATING BIOLOGICAL MATERIAL WITH ELECTROMAGNETIC RADIATION DELIVERED BY AN OPTICAL FIBER” filed on Apr. 19, 2001 and incorporated herein by reference.

### BACKGROUND OF THE INVENTION

Medical electromagnetic radiation treatment systems are used in a wide variety of medical applications. The optical characteristics of electromagnetic radiation of such treatment systems may provide more favorable results in comparison to traditional medical treatment tools. In addition, new medical procedures utilizing electromagnetic radiation have been developed that were not considered possible prior to the introduction of such medical electromagnetic radiation treatment systems.

It would be beneficial to further improve the current medical electromagnetic radiation treatment systems. It is, desirable, for instance, to improve the selectability of the impact of the output electromagnetic radiation such that only the target tissue is affected by the electromagnetic radiation while the healthy tissue is unharmed.

In fields such as optical communication networks it is known to use Graded Index (GRIN) optical fibers, for example, for efficiently transmitting electromagnetic signals from a source to a target.

It would be beneficial to have a medical electromagnetic radiation system which provided a beam more useful for performing a variety of medical procedures.

### SUMMARY OF THE INVENTION

The present invention provides a device and a method for treating the human body which includes generating electromagnetic radiation, for example, laser radiation, passing the electromagnetic radiation through a graded index optical fiber, the graded index optical fiber outputting the modified electromagnetic radiation, and applying the modified electromagnetic radiation to the human body.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a cross sectional view illustrating the transmission characteristics of a GRIN optical fiber according to an embodiment of the present invention.

FIG. 2 is a block diagram illustration of a medical electromagnetic radiation treatment system comprising a GRIN according to an embodiment of the present invention.

FIG. 3a is a graphic visualization of the distribution of a laser beam having a wavelength of 2.12 micrometers exiting a GRIN fiber, according to an embodiment of the present invention;

FIG. 3b is a graphic visualization of the distribution of a laser beam having a wavelength of 2.12 micrometer exiting a prior-art, step index fiber;

FIG. 4a is a photograph taken from above showing incisions made in turkey meat delivered from both a device according to an embodiment of the present invention and a prior art step index fiber;

FIG. 4b is a photograph taken from the side showing incisions made in turkey meat delivered from both a device according to an embodiment of the present invention and a prior art step index fiber;

FIG. 5 is a comparative illustration of the interaction between the output radiation of a Ho:Yag laser and water when the radiation is carried by and emitted from the step index fiber (left) of FIG. 3b versus a device according to an embodiment of the present invention; and

FIG. 6 is a flow chart depicting a series of steps according to an embodiment of the present invention

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

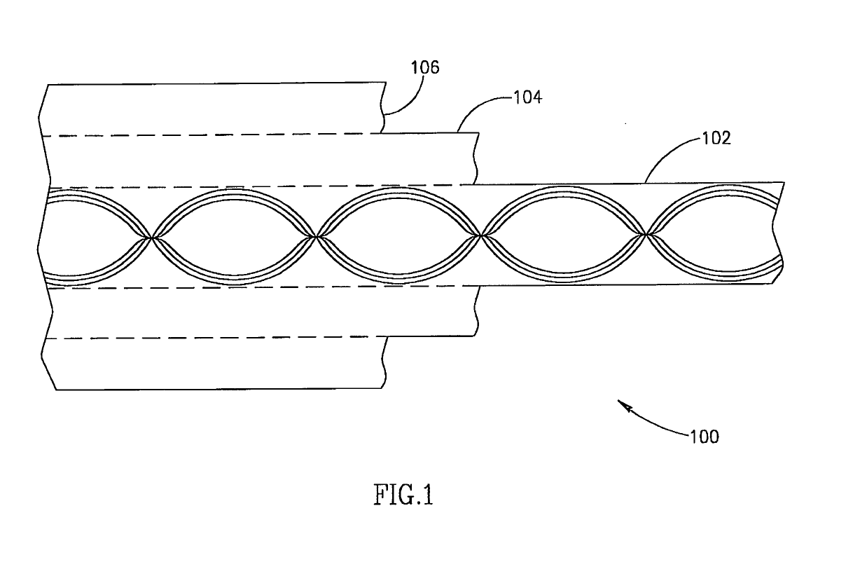

Turning to FIG. 1 there is shown a cross sectional view illustrating the transmission characteristics of a GRIN optical fiber which may be used with embodiments of the system and method of the present invention. The multimode GRIN fiber 100 may be comprised of, for example, a core having a refractive index that decreases, typically somewhat continuously, with increasing radial distance from the fiber axis 102, a clad 104 and one or more additional coatings 106. The clad 104 and the core 102 determine the electromagnetic transmission characteristics of the GRIN fiber 100. The clad 104 is not necessary, but may help to enhance transmission. The additional coating may be added for strength enhancement, to enhance optical characteristics, or for other reasons.

The core's 102 index of refraction is typically highest at the center, and is radially reduced to, typically, substantially equal the refractive index of the clad 104 at the boundary. This difference in the refraction index between the core and the clad is partially responsible for the electromagnetic radiation transmission characteristics. In alternate embodiments, the index of refraction at the edge of the core 102 to be reduced to that substantially equal to that of the clad 104. Various graded index core profiles suitable for use with embodiments of the present invention may be designed and implemented including, but not limited to, a parabolic like profile, a pyramid like profile and others. In other embodiments, a non-step refractive index function may be used as a core profile.

The graded index design of the core 102 causes the electromagnetic radiation entering the GRIN fiber 100 to be constantly refracted towards the center of the core. Due to the propagation characteristics of electromagnetic radiation along the GRIN fiber 100 the output radiation emitted from the GRIN fiber 100 may be modified such that the intensity distribution of the radiation is increased around the center when viewed cross-sectionally. For example, the output radiation may have an about Gaussian or Lorentzian intensity distribution, or more towards a Gaussian or Lorentzian distribution than that of the input beam. Other cross sectional distributions may be produced, such as a bell curve distribution, or a distribution more towards a bell curve than that of the input beam. The level of intensity of the radiation may depend upon, among other factors, the length of the GRIN fiber 100. A substantially Gaussian or Lorentzian intensity may be achieved provided that the length of the GRIN fiber 100 is above a certain threshold. In other embodiments, less than this threshold length may be used to move the intensity distribution towards the Gaussian or Lorentzian. The GRIN fiber 100 may be manufactured and designed using known methods.

According to embodiments of the present invention, the intensity distribution around the center of the output radiation exiting the GRIN fiber 100 is higher than the intensity distribution around the center of the input radiation entering the GRIN fiber 100. For example, the output radiation exiting the GRIN fiber may have a Gaussian intensity distribution. According to further embodiments of the present invention the intensity distribution of the output radiation exiting the GRIN fiber 100 is more symmetrical than the intensity distribution of the input radiation entering the GRIN fiber 100.

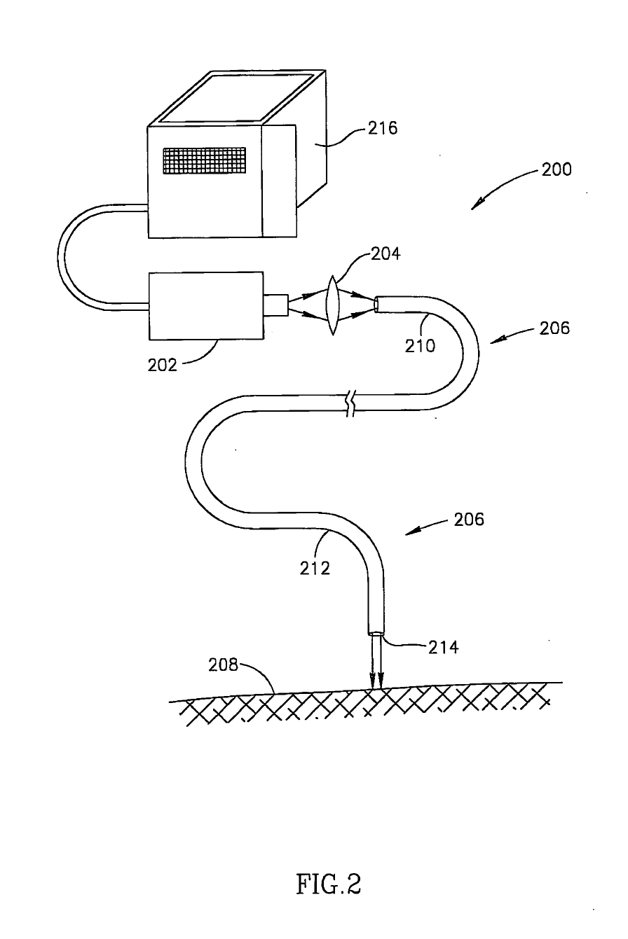

Reference is now made to FIG. 2 which is a block diagram illustration of a medical electromagnetic radiation treatment system according to an embodiment of the present invention. The medical electromagnetic treatment system 200 includes an illumination source 202 capable of producing electromagnetic radiation, such as a laser beam. The electromagnetic radiation generated by the illumination source 202 may be coupled to the fibers transmitting the beam to the tissues using any known optical coupling means 204, such as a focusing lens, to a guidance element 206 capable of transmitting the electromagnetic radiation, such that radiation generated by the illumination source 202 may be transported and directed onto a target tissue 208. In the embodiment shown in FIG. 1 the input portion 210 of the guidance element 206 is a step index optical fiber and the output portion 212 of the guidance element 204 is a GRIN optical fiber. In alternate embodiments, input portion 210 may be any sort of light guide, or maybe omitted. Portions 210 and 212 may be coupled using known methods. Typically, the output end 214 of the GRIN optical fiber 212 is located proximate to a biological tissue such that the radiation may be delivered directly to target tissue through the output end of the GRIN fiber 212. Portion 212 may be any fiber having variable refractance characteristics suitable for use in embodiments of the present invention.

In alternate embodiments, other structures and sets of elements may be used. For example, the step index optical fiber may be omitted. The radiation need not be delivered directly to the target tissue.

The term “delivered directly”, as used above with reference to delivering radiation form the GRIN fiber 212 to the biological material 208, means that the radiation proceeds from the exit end of the GRIN fiber 214 without passing through or being refracted form any optical component or components for focusing or manipulating the radiation. The radiation may pass through, for example, a layer of saline solution overlying the target tissue, or through another agent or an element which maybe in direct contact with the tissue.

In operation, electromagnetic radiation enters the input portion 210 as a laser beam having a substantially “top-hat” cross sectional intensity distribution, and exits the output portion 212 as a laser beam having a cross sectional intensity distribution which is more rounded than the input beam. For example the output beam may have an intensity distribution which is Gaussian, parabolic, bell curve shaped, or more Gaussian, parabolic or bell curve shaped, than the input beam.

According to one embodiment of the present invention the exit end 214 of the GRIN fiber 212 is in direct contact with the target tissue. The exit end 214 of the GRIN fiber 212 may be coated with a non-stick material such as Teflon or other suitable materials.

According to another embodiment of the present invention the targeted tissue is 208 irrigated with saline or any other solution and the output electromagnetic energy is applied onto the solution. The output end 214 of the GRIN fiber 212 may be in direct contact with the solution. The solution may be applied onto the target tissue 208 in accordance with any known or yet to be devised methods.

According to one embodiment of the present invention the GRIN optical fiber 212 may have a refractive index profile graded parabolically from a value of approximately 1.475 at the core center to a value of approximately 1.447 at the core's edge and the step index optical fiber 210 may have a refractive index value of approximately 1.447. However, other GRIN optical fibers 212 having different index profiles and/or values and other step index optical fibers 210 having a different refractive index may be selected.

The dimensions of the core of the GRIN optical fiber 212 may be dictated by, for example, the natural tolerance of the material selected for manufacturing the GRIN fiber 212 in accordance with any production methods known or yet to be devised, or by other factors such as the characteristics of the input beam or the desired characteristics of the output beam. In a typical embodiment, the GRIN optical fiber 212 has a core diameter of approximately between 150 and 1000 micrometers, although other diameters may be used. Using a typical embodiment focuses or tapers the electromagnetic radiation to a spot that is about 200 μm in diameter. However, spot sizes smaller or larger than 200 μm may be produced.

According to some embodiments of the present invention the illumination source 202 may be, for example, a CTH:YAG laser capable of generating a wavelength of approximately 2.12μm, a Th:YAG laser capable of generating a wavelength of approximately 2.0 μm, an Nd:Yag laser capable of generating laser radiation having a wavelength of approximately 1.06 μm or a Ho:Yag laser capable of generating laser radiation having a wavelength of approximately 2.1 μm (wherein, for example, approximately 2.1 μm includes, at least, 2.12 μm). Other illumination sources, generating beams with other characteristics, may be used. Preferably, the output wavelength or wavelengths generated by the illumination source 202 are within the wavelength tolerance of the GRIN optical fiber 212. However, according to other embodiments of the present invention, the illumination source 202 may be adapted to generate coherent or non-coherent electromagnetic radiation having a wavelength or wavelengths not within or only partially within the tolerance of the GRIN fiber 212.

According to yet another embodiment of the present invention the medical treatment system 200 may further comprise an optical filter or filters (not shown). The filters may be capable of filtering the electromagnetic radiation generated by the illumination source 212. The filters may be used for example, to filter out electromagnetic radiation having a wavelength or wavelengths that are outside the natural tolerance of the GRIN fiber 212, allowing all or a portion of the electromagnetic energy having a wavelength or wavelengths that is within the natural tolerance of the GRIN fiber 212. The filters may be used to tailor the electromagnetic radiation to the treatment desired.

According to some embodiments of the present invention the illumination source 202 may be operatively connected to a control module 216. The control module 216 may be adapted control the operation of the illumination source 202, including but not limited to switching between an active mode and an inactive mode, controlling the intensity of the radiation, controlling the duration of each active session, controlling the wavelength of the output radiation and pulsing the output of the illumination source 202. The control module may include known equipment used with electromagnetic radiation sources such as a power supply.

According to an embodiment of the present invention the GRIN fiber 212 has a length of at least 20 centimeters and the output radiation at the GRIN fiber's 212 exit end 214 is approximately Gaussian. In alternate embodiments, other lengths may be used. For example, a length of more or less than 20 cm may be used. A length shorter than that which substantially converts the electromagnetic beam to a having a Gaussian profile may be used. Depending on the characteristics of the GRIN fiber 212, different lengths may be used to produce the same change in the electromagnetic beam.

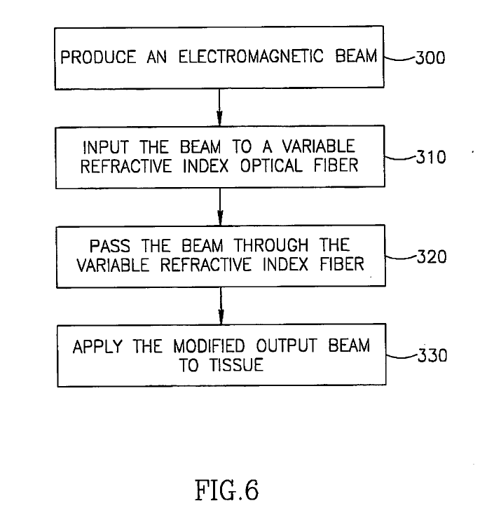

FIG. 6 is a flow chart depicting a series of steps according to an embodiment of the present invention. Referring to FIG. 6, in step 300, an electromagnetic beam is produced. Typically, the electromagnetic beam is a laser beam having a “top hat” intensity profile, but other types of beams having other profiles may be used.

In step 310 the beam is input to a variable refractive index optical fiber. In one embodiment, the optical fiber is a GRIN fiber, but other fibers may be used. The laser beam may be input to the optical fiber using any known method, for example the laser beam may be focused into the optical fiber using a focusing lens. Typically, before entering the fiber, this beam is modified to maintain the proper laser characteristics, for example, spot size, power, fluence, pulse duration, coherence, numerical aperture, such that a suitable fiber will accept its energy.

In step 320, the laser beam is passed through the variable refractive index fiber. The GRIN fiber may have a sufficient length to modify intensity distribution of the input electromagnetic radiation.

In step 330, the modified output beam is applied to tissue, for example, the human body, for treatment. Such treatment may include, for example, ablation or cutting, but other treatments may be used. The system and method of the present invention may be used, for example, for surgical procedures involving open, laparoscopic and endoscopic ablation, vaporization, excision, incision, and coagulation of soft tissue, or for other procedures. Various tissues and other organic and inorganic material in the body may be operated on, such as soft tissues, cartilage, bone, and other objects, stones to be fragmentated, stents and graphs. In alternate embodiments, the output may be applied to tissue other than human tissue.

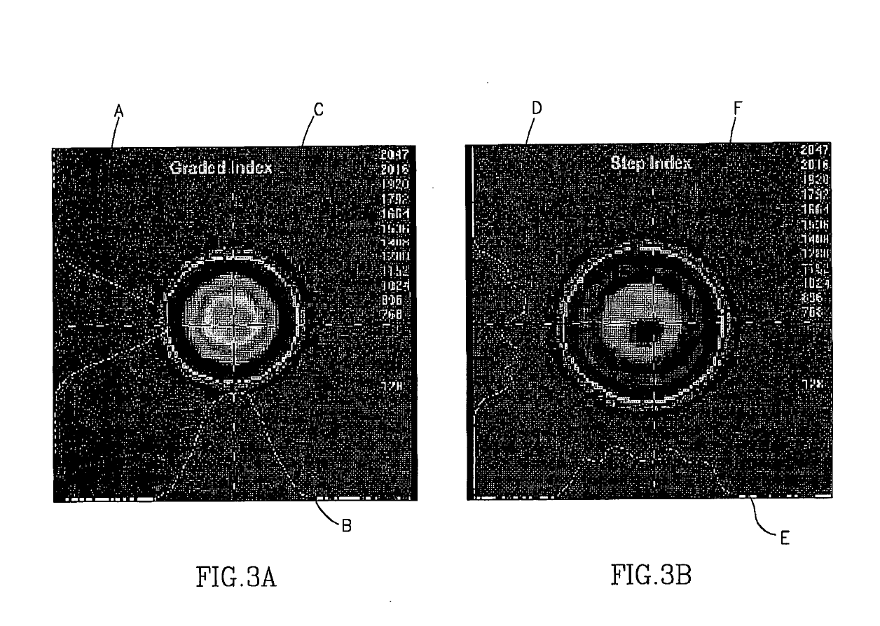

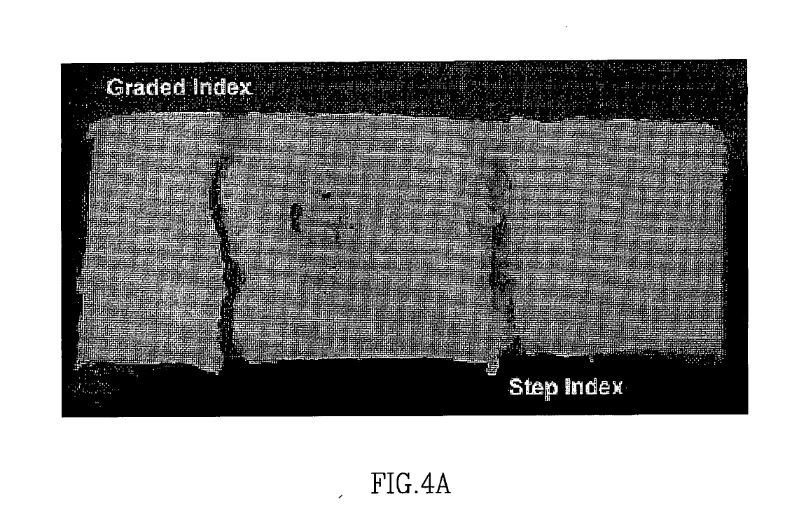

FIG. 3a graphically depicts the measured distribution of 2.12 μm wavelength laser radiation exiting a GRIN fiber, according to the operation of an embodiment of the present invention. In this example, the GRIN fiber has a core diameter of 600 μm and a refractive index graded parabolically from a value of 1.475 at the core center to 1.447 at the core edge. Curves A and B represent the intensity distribution of radiation along azimuths at 90 degrees to each other. The Gaussian form of the distribution is evident. Central image C shows the intensity of the laser radiation at the exit end of the GRIN fiber in contour form. The substantially circular form of the contours together with curves A and B indicate a relatively symmetrical distribution of radiation.

By way of comparison, FIG. 3b graphically depicts the measured distribution of 2.12 μm laser radiation exiting a prior art step index fiber. Curves D and E represent the intensity distribution of radiation along azimuths at 90 degrees to each other. Central image F shows the intensity at the exit end of the step index fiber in contour form. As is shown by curves D and E and image F the intensity distribution of the radiation at the exit end of the optical fiber is relatively uneven and asymmetric. Furthermore, the form of the uneven distribution may be expected to change with time or with movement of the fiber. The change in form may occur, for example, due to changes in the interaction of transmission modes of the laser radiation along the step index fiber.

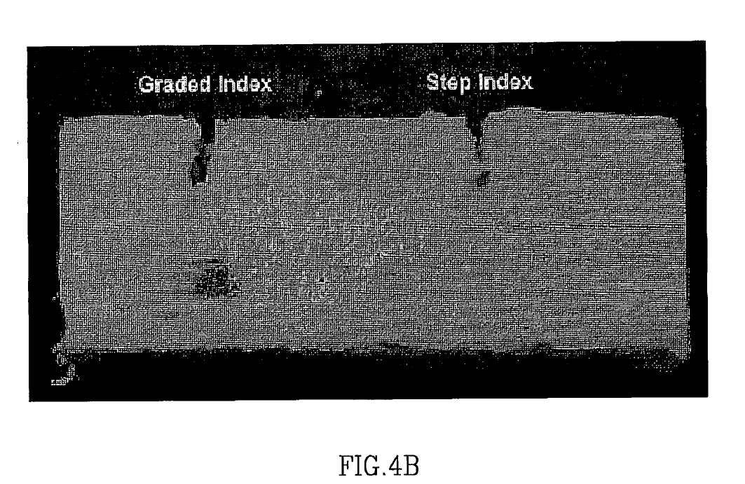

Turning now to FIGS. 4a and 4b showing photographs taken from above and from the side, respectively, of incisions made in turkey meat by radiation from the GRIN fiber of FIG. 3a (left) and from the prior art step index fiber of FIG. 3b (right). Power delivered by each of the fibers was 100 Watts at a wavelength of 2.12 μm. The fibers were dragged over the tissue by a motion control system while radiation was being delivered. The departure from straightness of the incisions is due to lateral movement of the fiber caused by an even surface of the turkey. Nevertheless, the image clearly shows that the incision made by the radiation delivered by the GRIN fiber clearly has cleaner, better defined edges and a more uniform width throughout than the incision made by the step index fiber. Various embodiments of the present invention may produce results that differ from the results shown.

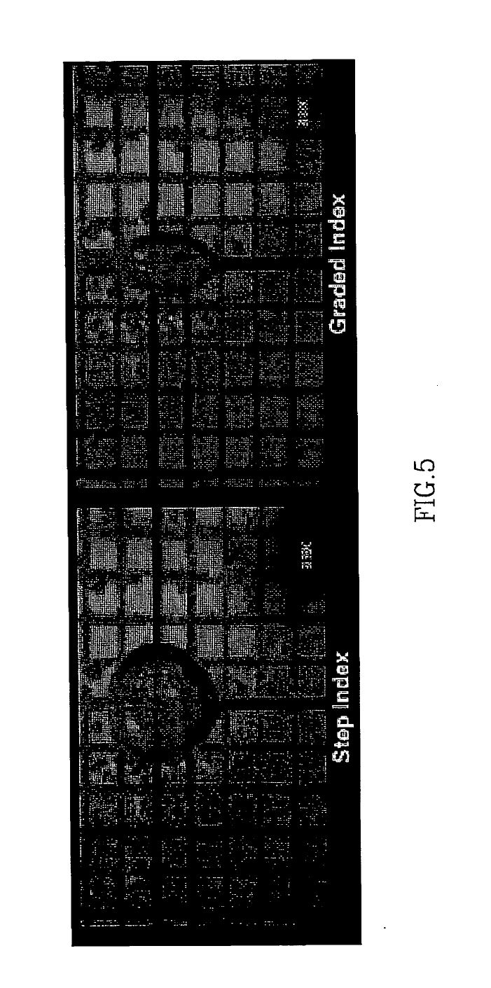

Turning now to FIG. 5 which is a photographic comparative illustration of the interaction between the output radiation of a Ho:Yag laser and water when the radiation is carried by and emitted from the step index fiber (left) of FIG. 3b versus a GRIN optical fiber of FIG. 3a having a sufficient length to convert the distribution of the radiation to an about Gaussian distribution (right). A step index fiber and GRIN fiber having a sufficient length to convert the distribution of the radiation to an about Gaussian distribution were provided, and the output end of each of the step index fiber and the GRIN fiber was placed in an aqueous environment. In the embodiment shown a 1.5 Joule pulse of Holmium laser was input to each of the step index fiber and the GRIN fiber. At the output end of each of the step index and the GRIN fiber a gas bubble was formed. This interaction between the Holmium and water or water solutions is known as the “Moses Effect”. The Moses Effect is undesirable since its formation is energy consuming such that less energy may be directed onto the target tissue. It was discovered the bubble formed using beams produced by devices according to an embodiment of the present invention has more of a torpedo like shape whereas the above described bubble formed at the exit end of the step index fiber has a spherical shape having a substantially larger volume. It is thus believed that less energy is wasted due to the “Moses Effect” when using a GRIN fiber having a sufficient length to convert the distribution of the radiation to an about gaussian distribution. In alternate embodiments of the present invention, other bubbles, having other shapes and sizes, may be produces. Of course, if the output from an embodiment of the present invention is not passed through liquid, no bubble may be formed.

It will be appreciated that the present invention is not limited by what has been described hereinabove and that numerous modifications, all of which fall within the scope of the present invention, exist. Rather the scope of the invention is defined by the claims, which follow.

1. A method of treating the human body, the method comprising:
producing electromagnetic radiation;
passing the electromagnetic radiation through a graded index optical fiber, the graded index optical fiber modifying the electromagnetic radiation and the graded index optical fiber outputting the modified electromagnetic radiation; and
applying the modified laser beam to the human body.

2. The method of claim 1 wherein said electromagnetic radiation is a laser beam.

3. The method of claim 2 wherein the laser beam has a wavelength of approximately 2.1 μm.

4. The method of claim 2 wherein the laser beam has a wavelength of approximately 1.06 μm.

5. The method of claim 1 comprising producing a laser beam by a CTH:YAG laser generator.

6. The method of claim 1 comprising producing a laser beam by a Ho:Yag laser generator.

7. The method of claim 1 wherein the modified electromagnetic radiation is applied directly to the human body.

8. The method of claim 7 comprising ablating the human tissue.

9. The method of claim 7 comprising cutting the human tissue.

10. The method of claim 1 comprising passing the electromagnetic radiation through a light guide.

11. The method of claim 1 comprising passing the electromagnetic radiation through a second optical fiber.

12. The method of claim 1 comprising passing the electromagnetic radiation through a lens.

13. The method of claim 1 wherein the modified electromagnetic radiation has a substantially Gaussian intensity distribution.

14. The method of claim 1 wherein the modified electromagnetic radiation has a substantially bell curve shaped intensity distribution.

15. The method of claim 1 comprising modifying the electromagnetic radiation in the graded index optical fiber such that the modified electromagnetic radiation has an intensity profile which is more rounded than that of the laser beam input to the fiber.

16. The method of claim 1 wherein the optical fiber has a core diameter of approximately between 150 and 1000 micrometers.

17. A method of treating the human body, the method comprising:
producing an original laser beam;
passing the laser beam through an optical fiber, the optical fiber outputting a modified laser beam, the modified laser beam having an intensity distribution which is more towards a Gaussian intensity distribution than the original laser beam; and
applying the modified laser beam to the human body.

18. The method of claim 17 wherein the laser beam includes a wavelength of approximately 2.1 μm.

19. The method of claim 17 wherein the laser beam includes a wavelength of approximately 1.06 μm.

20. The method of claim 17 wherein the modified laser beam is applied directly to the human body.

21. The method of claim 17 comprising ablating the human tissue.

22. The method of claim 17 comprising passing the laser beam through a lens.

23. The method of claim 17 wherein the modified laser beam has a substantially bell curve shaped intensity distribution.

24. The method of claim 17 comprising modifying the laser beam in a graded index optical fiber.

25. The method of claim 17 wherein the optical fiber has a core diameter of approximately between 150 and 1000 micrometers.

26. A method of treating the human body, the method comprising:
producing electromagnetic radiation;
passing electromagnetic radiation the through an optical fiber, the optical fiber having a continuously variable refractive index when measured from the center of the optical fiber to the edge of the optical fiber, the optical modifying the electromagnetic radiation, and the optical fiber outputting the modified electromagnetic radiation; and
applying the modified electromagnetic radiation to the human body.

27. The method of claim 26 wherein the electromagnetic radiation is a laser beam;

28. The method of claim 26 wherein the electromagnetic radiation has a wavelength of approximately 2.1 μm.

29. The method of claim 26 wherein the electromagnetic radiation has a wavelength of approximately 1.06 μm.

30. The method of claim 26 comprising ablating the human tissue.

31. The method of claim 26 wherein the modified electromagnetic radiation has a substantially bell curve shaped intensity distribution.

32. The method of claim 26 wherein the modified electromagnetic radiation has a substantially Gaussian intensity distribution.

33. The method of claim 26 comprising modifying the electromagnetic radiation such that the modified electromagnetic radiation has an intensity profile which is more rounded than that of the electromagnetic radiation input to the fiber.

34. The method of claim 26 wherein the optical fiber has a core diameter of approximately between 150 and 1000 micrometers.

35. A device for producing laser treatment for medical application on the human body, the device comprising:
an illumination source; and
a graded index optical fiber.

36. The device of claim 35 wherein said illumination source is a laser generatr.

37. The device of claim 35 comprising a lens.

38. The device of claim 35 comprising a second optical fiber.

39. The device of claim 35 wherein the illumination source is a CTH:YAG laser.

40. The device of claim 35 wherein the illumination source is a Ho:Yag laser.

41. The device of claim 35 wherein the illumination source produces at least a wavelength of approximately 2.1 μm.

42. The device of claim 35 wherein the illumination source produces at least a wavelength of approximately 1.06 μm.

43. The device of claim 35 comprising a second optical fiber.

44. The device of claim 35 wherein the electromagnetic radiation output from the graded index optical fiber has a substantially Gaussian intensity distribution.

45. The device of claim 35 wherein the electromagnetic radiation output from the graded index optical fiber has a substantially bell curve shaped intensity distribution.

46. The device of claim 35 wherein the electromagnetic radiation output from the graded index optical fiber has an intensity profile which is more rounded than that of the electromagnetic radiation input to the graded index optical fiber.

47. The device of claim 35 wherein the optical fiber has a core diameter of approximately between 150 and 1000 micrometers.
